(19) United States
(12) Patent Application Publication
(10) Pub. No.: US 2012/0059439 A1
(43) Pub. Date: Mar. 8, 2012

(54) ABERRATION CONTROL BY CORNEAL COLLAGEN CROSSLINKING COMBINED WITH BEAM-SHAPING TECHNIQUE

(75) Inventor: Geunyoung Yoon, Pittsford, NY (US)

(73) Assignee: University of Rochester, Rochester, NY (US)

(21) Appl. No.: 13/148,126

(22) PCT Filed: Feb. 12, 2010
(86) PCT No.: PCT/US10/24081

§ 371 Date: Oct. 4, 2011

Related U.S. Application Data
(60) Provisional application No. 61/151,956, filed on Feb. 12, 2009.

(51) Int. Cl. A61F 9/01; A61N 5/06; A61N 5/067; A61F 9/007

(52) U.S. Cl. 607/89; 607/90; 607/94

(57) ABSTRACT

Corneal collagen crosslinking is used to alter a characteristic of the cornea, such as thickness or refractive index, to correct wavefront aberrations, including higher-order aberrations. A scanning laser or the like is used to perform the corneal collagen crosslinking by locally altering the optical path length (thickness, refractive index, or both).

### REFERENCE TO RELATED APPLICATION

The present application claims the benefit of U.S. Provisional Patent Application No. 61/151,956, filed Feb. 12, 2009, whose disclosure is hereby incorporated by reference in its entirety into the present application.

### FIELD OF THE INVENTION

The present invention is directed to the correction of aberrations in the living eye and more particularly to such correction using corneal collagen crosslinking.

### DESCRIPTION OF RELATED ART

A minimally invasive and inexpensive treatment has been shown to stabilize the cornea in patients where keratectasia threatens vision. This treatment is called corneal collagen crosslinking (CXL or CCL). It is a technique that uses photochemical effects from riboflavin (vitamin B₂) excited with UV light to create extra intermolecular bonds in corneal collagen that increase its rigidity. Clinical studies show that the procedure can halt corneal regression without degrading vision. The technique is primarily being used as a conservative treatment for keratoconus, a disease where a conical bulge forms on the surface of the cornea and ruins the optical quality of the eye. Currently the procedure is approved in the European Union and is undergoing FDA trials in the Unites States. If long-term evidence proves its efficacy, CXL could offer keratoconus patients an early intervention option that would eliminate the any need for future corrective measures.

The cornea is the most powerful refracting surface in vision. Like all optical media in the eye, it must be highly transparent and uniformly curved. It is comprised of five distinct regions: the epithelium, the anterior elastic lamina, the stroma, the posterior elastic lamina and the endothelium. Of the regions, the stroma is the most important to CXL, as it contains a type I collagen matrix and constitutes the majority of corneal thickness. Within the matrix, millions of collagen fibers create the cornea's infrastructure. These fibers are formed by bundles of microfibrils running parallel to each other. Each microfibril is made up of numerous tropocollagen molecules, groups of three peptide chains wrapped around each other in a triple helix. Groups of collagen fibers arrange themselves in layers called lamellae. Every lamella is oriented orthogonal to its neighbor and parallel to the plane of the cornea. This organized structure maximizes the mechanical strength of the tissue while maintaining its transparency. The alternating orthogonal lamellae act as diffraction gratings and reduce the amount of light scattered by the tissue above it. Between the lamellae are sparse cells called keratocytes. They are responsible for a number of processes in the cornea that maintain its transparency and control wound healing. They are also known to secrete superoxide dismutase, an enzyme that blocks oxidizing agents (such as O₂⁻) from degrading corneal tissue. In cases where keratocytes do not function properly, the cornea's mechanical integrity can be compromised leading to a slew of complications that impair vision.

Many diseases and postoperative side effects can lead to a thinning and weakening of corneal tissue. Among the most frequent are keratoconus and post LASIK ectasia. Keratoconus is a disease with an onset at puberty that afflicts between 50 and 230 individuals out of 100,000. The disease is characterized by a conical protrusion that forms as the cornea becomes thin and weak, allowing intraocular pressures from the aqueous humor to push the anterior surface outward. Corneal topography maps of keratoconic corneas show an increase in K values towards the central region of the front surface. While peripheral K values might range between 40 and 45 D, the central region can range from 50 to 80 D depending on the severity of bulge. This irregular feature induces myopia and astigmatism in the eye and can severely impair vision. The underlying processes that weaken the cornea remain unknown; however, malfunctioning keratocytes are thought to contribute to the disease. The abnormally thin corneas of keratoconic patients are due to a reduced number of lamellae within the stroma. Within each lamella, there are also a reduced number of collagen microfibrils, causing a weakened infrastructure. Both these factors lead to corneal regression and require corrective measures to retain a patient's vision.

Classical treatments for keratoconus focus on correcting refractive errors but leave the underlying mechanisms of collagen degradation untreated. Different types of contact lenses are used to restore a patient's vision, including hard contact lenses for extreme corrections. Unfortunately, prolonged contact use often leads to corneal abrasions. The thin corneas and a reduced healing ability of keratoconics make this corrective method especially dangerous. Contact lenses have even been shown to accelerate corneal regression. A more promising management strategy is the implantation of small intracorneal rings called Intacs (Addition Technology Inc). They apply an elastic force that flattens corneal bulges by stretching the elevated regions out towards the periphery of the eye. Again, the success that Intacs have in patients with rapidly progressing keratoconus is limited since they do not address the root causes of corneal thinning. Prior to corneal CXL, the last resort for keratoconus patients was a keratoplasty. In 21% of all keratoconus cases, this is the only solution to restore vision. While the availability of donor corneas has improved, common risks of infection and transplant rejection remain. Recovery periods can also last up to 6 months and leave patients with impaired vision during that time.

In the late 1990's, a group in Dresden, Germany, began researching the application of a well-known materials science process to the treatment of keratoconus. This process was collagen fiber crosslinking. The team of researchers included three of the most prominent names in CXL, Gregor Wollensak, Theo Seiler and Eberhard Spoerl. They recognized how crosslinking had been used for years in dentistry to harden polymer materials and in bioengineering to stabilize collagen tissue. It effectively increases the elastic modulus of a collagen matrix by forming new molecular bonds between adjacent collagen microfibrils and within individual fibrils. With Michael Huhle they began researching the best way to create crosslinks in the type I collagen matrix of the cornea. After examining several mechanisms including various aldehyde chemical treatments, UV irradiation coupled with a photosensitizing agent was found to be the best means of inducing CXL in the cornea.

CXL with riboflavin and UV light is a photodynamic process that begins with the energetic excitation of riboflavin. Riboflavin or vitamin B₂ has three absorption peaks at 270, 366 and 445 nm. When irradiated with long UV light (typically 365-370 nm) the molecule enters its triplet state creating reactive oxygen molecules such as singlet oxygen (O²⁻). These free radicals then interact with amino acid groups to form bonds between adjacent collagen fibrils. Within a tropocollagen molecule, fibrils exist in a triple helix tail and are held together by hydrogen bonds. When oxygen radicals are introduced to this helix, they allow for stronger covalent bonds to form between each of the three fibrils. This also expands the collagen molecule itself and leads to an overall increase of the collagen fiber diameter. Also, these bonds make collagen more resilient to enzymatic digestion. Collagenase, for example, is an enzyme that degrades collagen fibers and has been linked to the progression of keratoconus. In a study performed by the Dresden group, they found that after CXL, collagen tissue was resistant to collagenase as well as pepsin and trypsin.

One of the most important CXL effects tested by researchers was the expected increase in corneal rigidity. Studies on porcine corneas had already confirmed the stiffening effect, but trials on human eyes had yet to be performed. Gregor Wollensak first quantified the biomechanical rigidity of crosslinked collagen in enucleated human corneas. Using UV-riboflavin CXL, his group treated corneal strips taken from five enucleated human eyes. For each eye, a control strip was retained to compare the crosslinking effect. A biomaterial tester measured the stress-strain values of the crosslinked and control corneal strips. It was shown that the human corneas became stiffer by 4.5 times after CXL, much higher than the 2 times increase observed in porcine eyes. This finding was a huge success and confirmed that the crosslinking principle could be applied to human corneas. However, several concerns remained regarding the safety of the procedure.

After several proof of concept experiments, work began to make UV-riboflavin crosslinking a clinically safe and effective procedure for treating keratoconus. Three side effects that had been observed to this point included keratocyte apoptosis, endothelial cell damage and a corneal haze that developed after crosslinking. However, the entire depth of the cornea was not subject to these effects. Instead, only the anterior portion of the stroma seemed to experience any negative side effects under a UV illumination of 3.0 mW/cm² and insulation of 0.1% riboflavin solution, This can be attributed to Beer-Lambert's law of absorption. As riboflavin absorbs UV light to create oxygen radicals, it reduces the intensity incident on collagen layers bellow it. Light intensity diminishes follows the equation: I_{depth}=I_{surface}e^{-dμ} is the absorption coefficient and d is the depth in the cornea. On one hand, this effect creates a safety depth in the CXL procedure by absorbing harmful UV rays before they reach sensitive regions of the eye including the endothelial layer of the cornea, the lens, and even the retina. On the other hand, it limits the amount of collagen that can be crosslinked. Researchers set out to find a middle ground; an irradiance that was powerful enough to create a significant amount of crosslinking while avoiding damage to important structures in the eye.

In previous studies, the anterior region of the cornea that was crosslinked had been swept clean of keratocytes. To address this issue, Wollensak, Spoerl, and Wilsch conducted two safety studies to quantify the dosage of UV light in the presence of riboflavin that led to keratocyte apoptosis. One study was an in vitro experiment that explored whether UV light, riboflavin or the combination of the two lead to keratocyte death. From this experiment, it was determined that the oxidizing agents released during crosslinking, not by the UV light itself caused cell death. In the second study, the eyes of 34 rabbits were exposed to varying intensities of UV light from 0.75 to 4 mW/cm². 4 and 24 hours following the crosslinking procedure, slices of the cornea were examined and the location of keratocytes was recorded. It was found that a dosage of 0.5 mW/cm² was enough to kill keratocytes where riboflavin was also present. According to the Beer-Lampert model mentioned above, this corresponded to a depth of 300 μm given a surface irradiance of 3 mW/cm². The eradication of keratocyte cells was clear evidence that crosslinking effects needed to limited to the stroma. It also acts as a natural indicator of the effective depth of the procedure. Even though crosslinking had been limited to the anterior region of the stroma, UV light still could reach the epithelium

A more dangerous side effect of corneal crosslinking than keratocyte depopulation is endothelial and retinal cell damage from UV light. While keratocytes repopulate a crosslinked region, damaging any of these structures results in permanent consequences. Again, Wollensak performed two studies, one in vitro to establish a benchmark toxic irradiance level and one in vivo to confirm these results in rabbits. From the in vitro study, an endothelial cytotoxic threshold irradiance of 0.35 mW/cm² was found in the presence of riboflavin and a surface irradiance of 3 mW/cm². The same cytotoxic threshold was 4 mW/cm² for UVA light alone. Again, by the Beer Lampert approximation, this would occur at a corneal depth of 400 μm. In vivo results confirmed the cytotoxic UV levels observed in vitro. From these results, it was suggested that UV-riboflavin CXL not be performed on patients with cornea's thinner than 400 μm. This would reduce the risk of endothelial cell damage due to reactive oxygen produced by the procedure.

These four studies set the benchmarks for UV irradiance levels administered on human corneas. The risks of exposing the eye directly to UV light had been explored and successfully managed by establishing limits to the UV surface dosage (3.0 mW/cm²) and minimum corneal thickness (400 μm) over which CXL should be performed. Shortly after 2003, a number of clinical trials of UV-riboflavin CXL began in Europe (although preliminary trials had been taking place as early as 1998).

Each clinical study employed some slight variation of the standard UV-riboflavin CXL procedure understood to minimize UV damage. It begins with the mechanical removal of the corneal epithelium. Typically a 7-9 mm diameter circle is marked on the epithelium and then scraped away with a blunt spatula. This protective layer of the cornea inhibits the absorption of riboflavin into the stroma. As shown by Wollensak, riboflavin not only enables the crosslinking effect but also acts as a UV shield, protecting other structures from damage. The best shielding effects are realized when riboflavin diffuses throughout the full thickness of the stroma. In a recent study, riboflavin concentrations in the cornea were measured after a topical application with and without the epithelium in situ. With the epithelium in place, riboflavin concentrations were 100 times less than with the epithelium removed. Despite the discomfort and risk of infection from epithelial debridement, it is regarded as a necessary step to ensure the safety and efficacy of CXL.

Following the removal of the epithelium, a solution of 0.1% riboflavin and 20% Dextran is instilled into the cornea. The amount of riboflavin solution administered has varied significantly amongst research groups. In the first CXL procedures, one drop of riboflavin was administered to the cornea 5 minutes before UV illumination. Additional drops were added later. Now, most publications describe a treatment where there is an initial soaking period of 30 minutes prior to UV exposure when riboflavin is given the chance to diffuse completely throughout the cornea. This modified protocol began around the time Spoerl et al. published a theoretical model of diffusion dynamics of riboflavin in the cornea after epithelial debridement. Between 5 and 30 minutes after riboflavin exposure, its concentration in the first 50 μm of the cornea only increases from 0.08% to 0.09%. However, the concentration of riboflavin deep (400 μm) in the cornea quadruples from >0.01% to 0.04%¹³. This is reason to allow the riboflavin time to soak into the cornea before UV irradiation. Today, 1-3 drops of riboflavin are administered every 3 minutes for 30 minutes prior to any UV exposure.

Once riboflavin has been absorbed into the cornea, the UV treatment can begin. 360-370 nm has been used to excite the photochemical release of reactive oxygen elements from riboflavin. The other absorption peaks are not accessed since 270 nm light can damage the DNA of local cells and 445 nm irradiation can cause “blue light” damage to the retina. Typical sources used for clinical CXL are UV LED systems. They have multiple LED heads (5-7) which attempt to deliver a uniform light intensity profile. Originally, two headed UV sources were used but had the downside of creating uneven intensity profiles that caused dangerous irradiances over a given region, Prior to the procedure, UV sources are calibrated, as determined by Wollensak, to 3 mW/cm². Again, this provides effective CXL up to 300 μm into the cornea without damaging the endothelium. For the next 30 minutes, the cornea continues to be exposed to UV light while receiving 1-3 riboflavin drops every 5 minutes. The 30 minute exposure time delivers a total of 5.4 J/cm² to the front surface of the cornea. Following UV exposure, the cornea is instilled with medicated drops and artificial tears and is dressed with a soft contact lens. All three measures prevent against infection and irritation to the exposed anterior elastic layer.

In 2002, the Dresden group published the first clinical trials of corneal CXL performed on keratoconic patients with moderate to extreme keratectasia. The 22 patients enrolled in the study had documented cases of increasing maximum K values. Each patient's corneal topography, best corrected visual acuity, intraocular pressure, endothelial cell density and central corneal thickness was measured before the operation. One eye in each patient was selected to undergo CXL while the other was kept as a control. After the CXL procedure, follow-up examinations, over an average of two years, measured changes in the pre-operative parameters. It was found that the maximum K values had decreased by an average of 2D and visual acuity increased by slightly more than 1 line⁷. In the contralateral eye, 5 of the 23 eyes demonstrated a continued progression of keratoconus as K values increased by 1.48 D after 1 year following the procedure. The results were promising. For the first time, CXL had been used to halt the progression of keratoconus with a minimally invasive surgery. Best of all, the observed complications of the procedure were nearly non-existent. The major concerns of UV induced endothelial and lens damage had been avoided. Endothelial cell counts remained unchanged post-operatively and there were no signs of developing cataracts in the years following the procedure.

Another concern with vision, and one that has traditionally been seen as separate from the above, is the correction of wavefront aberrations in the human eye. Surgical techniques have long been used in the art to correct such wavefront aberrations. Moreover, while traditionally only up to second-order aberrations have been corrected by such techniques, higher-order wavefront aberrations are increasingly understood as important to vision. Higher-order aberrations can include at least third-order aberrations, at least fourth-order aberrations, at least fifth-order aberrations, and even up to tenth-order aberrations. Techniques for detecting and correcting such aberrations are disclosed in U.S. Pat. No. 5,777,719, whose disclosure is hereby incorporated by reference into the present disclosure.

The following references supplement the above description:
Meek K M, Leonard D W. Ultrastructure of the corneal stroma: a comparative study. Biophys. J 1993; 64:273-280.
Maurice D M. The structure and transparency of the corneal stroma. J. Physiol 1957; 136:263-286.
Rabinowitz YS. Keratoconus. Surv Ophthalmol 1998; 42: 297-319.
Spoerl E, Huhle M, Seiler T. Induction of cross-links in corneal tissue. Exp Eye Res 1998; 66:97-103
Wollensak G. Crosslinking treatment of progressive keratoconus: new hope. Curr Opin Ophthalmol 2006; 17:356-360.
Spoerl E, Wollensak G, Seiler T. Increased resistance of crosslinked cornea against enzymatic digestion. Curr Eye Research 2004; 29: 35-40
Wollensak G, Sporl E, Seiler T. Treatment of keratoconus by collagen crosslinking. Ophthalmologe 2003; 100: 44-49.
Wollensak G, Spoerl E, Reber F, Seiler T. Keratocyte cytotoxicity of riboflavin/UVA treatment in vitro. Eye 2004; 18:718-722
Wollensak G, Spoerl E, Wilsch M, Seiler T. Keratocyte apoptosis after corneal collagen crosslinking using riboflavin/UVA treatment. Cornea 2004; 23:43-49
Wollensak G, Spoerl E, Reber F, Pillunat L, Funk R. Corneal endothelial cytotoxicity of riboflavin/UVA treatment in vitro
Wollensak G, Spoerl E, Wilsch M, Seiler T. Endothelial cell damage after riboflavin-ultraviolet-A treatment in the rabbit. J Cataract Refract Surg 2003; 29:1786-1790
Baiocchi S, Mazzotta C, Cerretani D, Caporossi T, Caporossi A. Corneal crosslinking: riboflavin concentration in corneal stroma exposed with and without epithelium. J Cataract Refract Surg 2009; 35:893-899
Spoerl E, Mrochen M, Sliney D, Trokel S, Seiler T. Safety of UVA-Riboflavin Cross-Linking of the Cornea. Cornea 2007; 26:385-389
Caporossi A, Baiocchi S, Mazzotta C, Traversi C, Caporossi T. Parasurgical therapy for keratoconus by riboflavin-ultraviolet type A rays induced cross-linking of corneal collagen; preliminary refractive results in an Italian study. J Cataract Refract Surg 2006; 32:837-845
Hafezi F, Kanellopoulos J, Wiltfang R, Seiler T. Corneal collagen crosslinking with riboflavin and ultraviolet A to treat induced keratectasia after laser in situ keratomileusis. J Catanc Rtelnet Sutg 20073:3:2035-20
Koller T, Mrochen M, Seiler T. Complications and failure rates after corneal crosslinking. J Cataract Refract Surg 2009; 35:1358-1362

### SUMMARY OF THE INVENTION

It is therefore an object of the invention to apply CCL to the correction of wavefront aberrations in the eye.

It is another object of the invention to apply CCL optionally to the correction of higher-order wavefront aberrations in the eye.

To achieve the above and other objects, the present invention is directed to a system and method for using the CCL procedure's thickness and/or refractive index change as a corrective procedure for such aberrations.

The inventor proposes to test the concept by treating enucleated pig eye corneas in accordance with the currently preferred corneal collagen crosslinking protocol: first, the central 7-9 mm of corneal epithelium is removed. Then, the cornea is pre-treated with a riboflavin solution (0.1%) for a period of 20-30 minutes, reapplying the solution every 3-5 minutes to ensure riboflavin penetration throughout corneal thickness. This is followed by irradiation with an ultraviolet light source with a wavelength of 365-370 nm and power of 3 mW/cm² is done for 30 minutes, with continued reapplication of the riboflavin solution every 3-5 minutes throughout the 30 minute treatment period. To investigate the thickness change caused, we will use each cornea as its own control: Using a physical obstruction of the light path, we will block half of the cornea from the UV exposure. Thus, the entire cornea will experience some swelling from absorption of the riboflavin solution, but only the unobstructed side will experience the collagen crosslinking effect. Then, we will perform analysis of the cornea using OCT measurement and a Shack-Hartmann wavefront sensor to examine the differences between the treated and untreated halves of the corneas. This is to determine the immediate effect of the collagen crosslinking procedure itself on corneal thickness, whether thickness increases or decreases, and by how much.

Investigation of the feasibility of the Corneal Collagen Crosslinking Procedure to induce higher order aberrations: Next, we aim to investigate whether we can control the thickness/refractive index change examined in the first aim in a predictable manner. Standard therapeutic procedure for corneal collagen crosslinking is detailed above, and all studies into corneal crosslinking have treated the entire cornea evenly using this procedure. In this aim we will look at how the elements of the procedure, such as time of irradiation, intensity of irradiation, and riboflavin solution concentration, can be varied so as to produce predictable local thickness/refractive index change. This will be investigated in an enucleated pig eye model. The second part of this aim will be moving our initial findings on predictability and controllability to a more advanced control system. Depending on the variable with which we have the most success in terms of predictability and controllability in the first part of this aim, we will attempt to induce higher order aberrations. To do this, we will design a computer controlled ultraviolet projection system which will, based on wavefront measurements, attempt to use our thickness change investigation findings above to try and vary treatment across the wavefront map to generate higher order aberrations in an enucleated pig eye model.

### BRIEF DESCRIPTION OF THE DRAWINGS

A preferred embodiment will be set forth in detail with reference to the drawings, in which:

FIG. 1 is a schematic diagram of a device on which the present invention can be implemented;

FIGS. 2A and 2B show an experimental setup used to test the concept; and

FIG. 3 shows plots of experimental results taken with the setup of FIGS. 2A and 2B.

### DETAILED DESCRIPTION OF THE PREFERRED EMBODIMENT

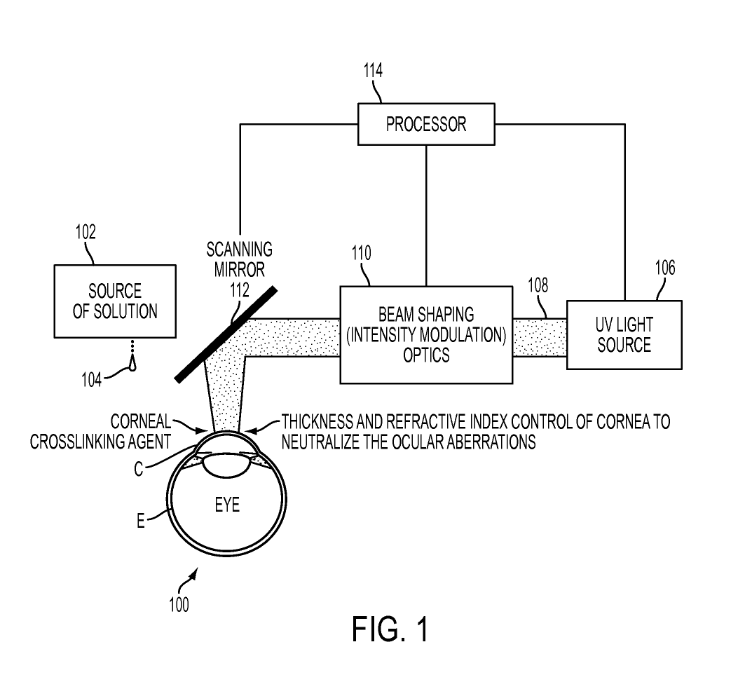

FIG. 1 shows a system 100 for use on the cornea C of a living human or other eye E. A source 102 of riboflavin solution 104 is used to apply the riboflavin solution to the cornea. Then a laser or other UV light source 106 emits a beam 108 of laser light at the cornea via beam shaping (e.g., intensity modulation) optics 110 and a scanning mirror 112 to perform corneal collagen crosslinking. The distribution of laser light provided by the laser (e.g., exposure time at a particular location, intensity at a particular location, or both) is calculated to correct higher-order wavefront aberrations that have been detected as taught, e.g., in the above-cited U.S. Pat. No. 5,777,719. Alternatively, only lower-order aberrations (through second order), or any orders or combinations of orders, may be corrected. Once the aberrations have been detected, as taught in the above-cited '719 patent or in any other suitable manner, the system 100 can be controlled automatically by a processor 114.

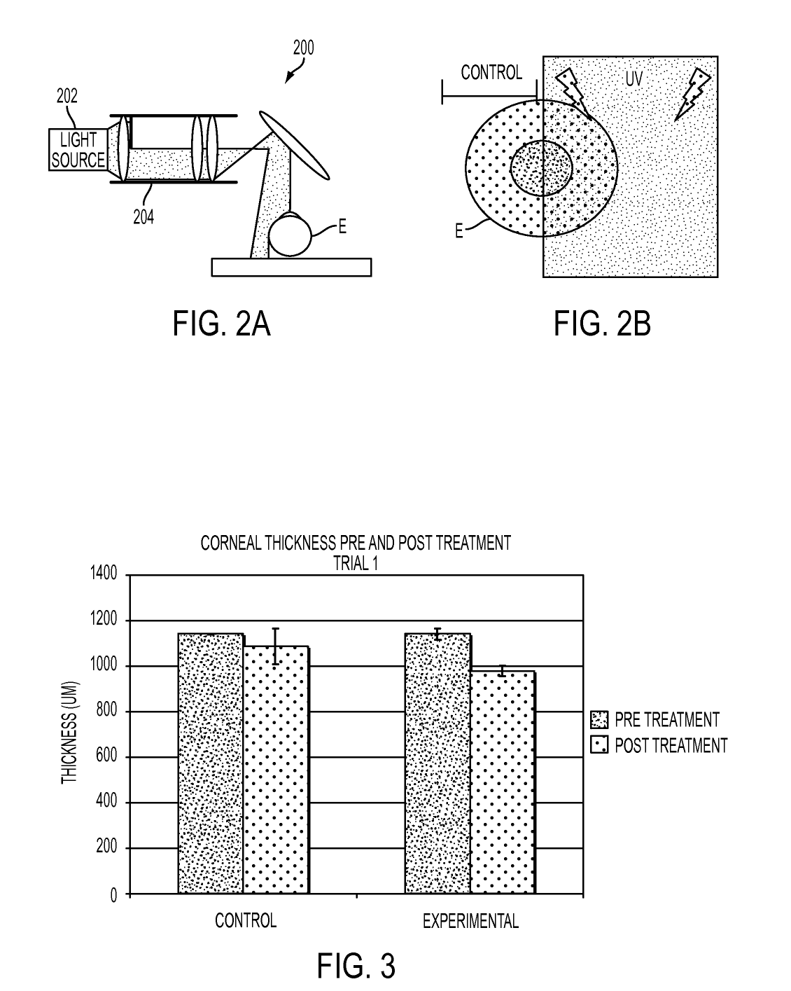

An experiment was performed using the setup 200 of FIG. 2A to test the concept. The central 6.5-9 mm of the epithelium of an eye E was removed with a blunt spatula. A riboflavin 0.1% solution was used, and the eye was pretreated with the solution every 2-5 minutes for 20-30 minutes. Ultraviolet light at 307 nm and 3 mW/cm² from a light source 202 was applied to the eye through optics 204 that occluded half of the eye to create a control, as shown in FIG. 2B. Four eyes were thus treated and measured.

FIG. 3 shows preliminary results for the control (left) and UVA illumination (right). The thinning effect was more marked after UVA illumination than with the control. The averaged corneal thinning of the four eyes was 126±92 μm.

While a preferred embodiment has been set forth above, those skilled in the art who have reviewed the present disclosure will readily appreciate that other embodiments can be realized within the scope of the invention. For example, numerical values are illustrative rather than limiting. Also, a human or non-human eye may be treated. Moreover, any suitable device for applying the correct radiation to the eye to correct the wavefront aberrations may be used, including without limitation a scanning laser, a beam shaper, a deformable mirror, or any combination of such elements. Therefore, the invention should be construed as limited only by the appended claims.

I claim:

1. A method for correcting wavefront aberrations in a living eye, the method comprising:
(a) illuminating the cornea to cause corneal collagen crosslinking; and
(b) controlling the corneal collagen crosslinking such as to change a characteristic of the cornea to correct the aberrations.

2. The method of claim 1, wherein the characteristic comprises thickness.

3. The method of claim 1, wherein the characteristic comprises index of refraction.

4. The method of claim 1, wherein the aberrations comprise higher-order aberrations.

5. The method of claim 4, wherein the aberrations comprise at least third-order aberrations.

6. The method of claim 5, wherein the aberrations comprise at least fourth-order aberrations.

7. The method of claim 6, wherein the aberrations comprise at least fifth-order aberrations.

8. The method of claim 1, wherein step (a) comprises treating the cornea of the eye with a riboflavin solution.

9. The method of claim 1, wherein step (a) comprises using a scanning laser, and wherein step (b) comprises controlling the scanning laser to cause local changes in the characteristic.

10. The method of claim 1, wherein step (a) is performed using UVA light.

11. A system for correcting wavefront aberrations in a living eye, the system comprising:
a light source for illuminating the cornea to cause corneal collagen crosslinking; and
a processor for controlling the light source to control the corneal collagen crosslinking such as to change a characteristic of the cornea to correct the aberrations.

12. The system of claim 11, wherein the light source and the processor are configured such that the characteristic comprises thickness.

13. The system of claim 11, wherein the light source and the processor are configured such that the characteristic comprises index of refraction.

14. The system of claim 1, wherein the light source and the processor are configured such that the aberrations comprise higher-order aberrations.

15. The system of claim 14, wherein the aberrations comprise at least third-order aberrations.

16. The system of claim 15, wherein the aberrations comprise at least fourth-order aberrations.

17. The system of claim 16, wherein the aberrations comprise at least fifth-order aberrations.

18. The system of claim 11, further comprising a source of riboflavin solution for treating the cornea of the eye with the riboflavin solution.

19. The system of claim 11, wherein the light source comprises a scanning laser, and wherein the processor controls the scanning laser to cause local changes in the characteristic.

20. The system of claim 11, wherein the light source emits UVA light.
